(19) United States
(12) Patent Application Publication
(10) Pub. No.: US 2016/0207879 A1
(43) Pub. Date: Jul. 21, 2016

(54) METHODS AND INDUSTRIAL PROCESSES FOR THE GENERATION OF FREE FATTY ACIDS AND DERIVATIVES THEREOF FROM OIL GUMS

(71) Applicant: INVENTURE RENEWABLES, INC., Tuscaloosa, AL (US)

(72) Inventors: William W. BERRY, Tuscaloosa, AL (US); William Rusty SUTTERLIN, Tuscaloosa, AL (US); Mark G. TEGEN, Tuscaloosa, AL (US); Cory BLANCHARD, Tuscaloosa, AL (US)

(21) Appl. No.: 14/913,608

(22) PCT Filed: Aug. 29, 2014
(86) PCT No.: PCT/US14/53599

§ 371 Date: Feb. 22, 2016

Related U.S. Application Data
(60) Provisional application No. 61/872,446, filed on Aug. 30, 2013.
(60) Provisional application No. 61/990,618, filed on May 8, 2014.

(51) Int. Cl. C07C 231/10; C11C 3/00

(52) U.S. Cl. CPC C07C 231/10; C11C 3/00

(57) ABSTRACT

This invention generally relates to the generation of free fatty acids and/or derivatives thereof, e.g. fatty alkyl esters, and/or fatty amides, from feedstocks comprising phospholipids. In alternative embodiments, the invention provides methods and industrial processes for generating free fatty acids and/or fatty acid derivatives from a feedstock comprising phospholipids by combining the feedstock with one or more reactants to form a reaction mixture and reacting the mixture at a temperature and pressure sufficient to provide for the separation of the free fatty acids from their phosphatide structures.

### RELATED APPLICATIONS

This Patent Convention Treaty (PCT) International Application claims the benefit of priority under 35 U.S.C. §119(e) of U.S. Provisional Application Ser. No. 61/872,446, filed Aug. 30, 2013, and U.S. Ser. No. 61/990,618, filed May 8, 2014. The aforementioned applications are expressly incorporated herein by reference in their entirety and for all purposes.

### FIELD OF THE INVENTION

This invention generally relates to the processing of feedstocks comprising phospholipids, e.g., phosphatidylcholine, phosphatidylethanolamine, phosphatidylinositol, phosphatidic acid, to generate free fatty acids and their derivatives, fatty alkyl esters, glycerol, glycerol derivatives, e.g. glycerol choline phosphate, fatty amides, fatty amines or related compounds. In alternative embodiments, the inventions provides methods and industrial processes for generating free fatty acids and free fatty acid derivatives e.g. alkyl esters, fatty amides, fatty amines, glycerol and/or glycerol derivatives, or a combination thereof, comprising directly contacting a phospholipid material with a fluid in a pressurized reactor whereby the partial pressure of the system provides for solvolysis, e.g. hydrolysis, alcoholysis (i.e. esterification), ammonolysis or a combination thereof, and/or acidification.

### BACKGROUND OF THE INVENTION

Crude glyceride animal or vegetable oils typically undergo several refining steps in order to remove fatty acids, phospholipids, and other impurities in order to generate a consumable finished product. The first stage of the refining process, referred to as “degumming”, involves the removal of phospholipids and other impurities from the crude oil in order to prevent gum buildup during oil storage. Gums can be removed from crude oils by hydrating them with water and removing the hydrated gums from the oil. Certain fruit oils cannot be degummed with water because the oils have already come in contact with water during the production process. These oils must be degummed chemically, resulting in a slightly different chemical make-up of the gums.

The composition of oil gums varies based on the starting material to be refined. Gums are comprised mainly of phospholipids, including phosphatidylcholine, phosphtidylethanolamine, phosphatidylinositol, phosphatidic acid, as well as various other lipid and lipid-derived impurities such as mono-, di-, and triglycerides, and free fatty acids. Oil gums are typically non-uniform across batches, are highly viscous, contain a number of different lipid and lipid-derived products in addition to phospholipids, and are not produced in large volumes. Due to these characteristics, gums do not lend themselves to conventional refining processes that depend on uniform feedstocks at large scales.

Currently, gums are either considered a waste stream with limited commercial value or marketed as a lecithin product for use as emulsifiers in various food products. Despite their chemical makeup, the economic conversion of gums into higher-value chemicals and fuels has been inhibited by 1) relatively small quantities of gums available per oil processing facility, making the economies of scale needed for most commercial processes to operate efficiently difficult to achieve, and 2) a lack of uniformity across gum feedstocks that the majority of industrial processes for lipid refining are unable to handle.

If further processed into discreet product streams, oil gums would possess significant commercial value beyond the value currently derived from an unrefined or semi-refined product. For example, if cleaved from their glycerol-phosphate structure, free fatty acids and/or free fatty acid derivatives, e.g. fatty acid alkyl esters, fatty amides, and fatty amines, could be marketed at a significant premium to raw or semi-refined gums.

### SUMMARY OF THE INVENTION

In alternative embodiments, the present invention provides methods and processes for the production of free fatty acids and/or derivatives thereof from a lipid-containing feedstock comprising phospholipids and, optionally other lipids, e.g. glycerides (acylglycerols). In alternative embodiments, the methods and processes comprise providing a reaction mixture that comprises the feedstock and a solvent and, optionally carbon dioxide (CO₂), and reacting the mixture at a temperature and pressure and for a time that is sufficient to provide for the solvolysis (e.g., an alcoholysis or ammonolysis) and/or acidification of substantially all of the phospholipids and, if present, other lipids in the feedstock.

In alternative embodiments, the invention provides methods and processes for the production of free fatty acids from lipid-containing feedstocks comprising phospholipids and, optionally, other lipids, e.g. glycerides (acylglycerols). The process comprises providing a reaction mixture that comprises the feedstock, a solvent such as water, and optionally CO₂, wherein the water and CO₂, if present, react to form carbonic acid, and reacting the reaction mixture at a temperature and pressure and for a time that is sufficient to provide for the solvolysis and/or acidification of substantially all of the phospholipids and, if present, the other lipids in the feedstock.

In alternative embodiments, the invention provides methods and processes for the production of fatty amides from lipid feedstocks comprising phospholipids and, optionally, other lipids, e.g. glycerides (acylglycerols). The process comprises providing a reaction mixture that comprises the feedstock, ammonia, and a catalyst, and reacting the reaction mixture at a temperature and pressure and for a time that is sufficient to provide for the solvolysis of substantially all of the phospholipids and, if present, the other lipids in the feedstock.

In alternative embodiments, the invention provides methods and processes for the production of fatty amides from lipid feedstocks comprising phospholipids and, optionally, other lipids, e.g. glycerides. The process comprises providing a reaction mixture that comprises the feedstock and a primary amine or a secondary amine, and optionally, a catalyst, and reacting the reaction mixture at a temperature and pressure and for a time that is sufficient to provide for the solvolysis of substantially all of the phospholipids and, if present, the other lipids in the feedstock.

In alternative embodiments, the invention provides methods and processes for the production of free fatty acids and/or free fatty acid derivatives, from a lipid-containing feedstock comprising phospholipids comprising the steps of:

(a) providing a feedstock comprising phospholipids, wherein optionally the feedstock further comprises non-phospholipid lipids (or, lipids other than phospholipids), and optionally the non-phospholipid lipids comprise glycerides, and optionally the glycerides are triglycerides;

(b) providing a reaction mixture comprising the feedstock and a solvent and, optionally, CO₂; and

(c) reacting the reaction mixture at a temperature, pressure and for a time that is sufficient to provide for the solvolysis, esterification, transesterification, acidification, or any combination thereof, of substantially all of the phospholipids and, if present, the lipids in the feedstock,

wherein optionally the time is between about 0 to about 24 hours, between about 0 to about 12 hours, e.g. between about 1 hour and about 8 hours, between about 3 hours and 5 hours, between about 5 minutes and one hour, between about 2 minutes to about 20 minutes, or between about 0 minutes to about 1 minute,

wherein optionally the solvolysis is an alcoholysis, an ammonolysis, a hydrolysis, or a combination thereof.

In alternative embodiments, the feedstock comprises from between about 1 to 10 wt % to about 90 to 100 wt % phospholipids, or from between about 10 to 20 wt % to about 80 to 100 wt % phospholipids, about 30 wt % to about 90 wt %, about 40 wt % to about 80 wt %, or about 50 wt % to about 75 wt % phospholipids.

In alternative embodiments, the feedstock further comprises between about 5 wt % to about 40 wt % free fatty acids, and between about 5 wt % to about 50 wt % glycerides (acylglycerols).

The feedstock can be an oil gum derived from a crude vegetable oil, or a mixture of oil gums derived from multiple vegetable oils. In alternative embodiments, the gum is derived from a crude vegetable oil selected from the group consisting of soybean oil, palm oil, canola oil, rapeseed oil, wheat germ oil, safflower oil, peanut oil, cottonseed oil, sunflower oil, grape seed oil, jatropha oil, palm kernel oil, coconut oil, olive oil, corn oil, hazelnut oil, linseed oil, rice bran oil, safflower oil, sesame oil, algae oil and a combination thereof.

In alternative embodiments, the reaction temperature is between about 10° C. to about 175° C., the pressure of the reaction is between about 0 psi and about 2500 psi, or between about 500 psi and about 2000 psi, between about 800 psi and about 1750 psi, or about 1000 psi and about 1500 psi, or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi, and the time of the reaction is between about 0 to about 12 hours.

In alternative embodiments, the solvent is water. The reaction mixture can comprise a CO₂ that reacts with the water to form carbonic acid in the reaction mixture. In alternative embodiments, the amount of water in the reaction mixture is between about 1 mol to about 100 mol per mol of feedstock, or between about 10 mol to about 70 mol per mol of feedstock, or about 30 mol to about 50 mol per mol of feedstock. The amount of CO₂ in the reaction mixture can be between about 1 mol to about 10 mol per mol of feedstock.

In alternative embodiments, the reaction mixture further comprises an alcohol, and wherein the alcohol esterifies at least a portion of the lipids in the feedstock to generate fatty alkyl esters. The amount alcohol in the reaction mixture can be between about 1 mol to about 100 mol per mol of feedstock, or between about 10 mol to about 70 mol per mol of feedstock, or about 30 mol to about 50 mol per mol of feedstock. The alcohol can contain between 1 and 9 carbon atoms, or the alcohol is a methanol, an ethanol, a butanol, an isopropyl alcohol, a sec-butanol, a t-butanol, a benzyl alcohol, or a combination thereof.

In alternative embodiments, the solvent is ammonia. The amount of ammonia in the reaction mixture can be between about 1 mol to about 100 mol per mol of feedstock, or between about 10 mol to about 70 mol per mol of feedstock, or about 30 mol to about 50 mol per mol of feedstock.

In alternative embodiments, the reaction mixture further comprises a, or at least one, catalyst. The catalyst can comprise an ammonium salt; or, the catalyst can be selected from the group consisting of ammonium nitrate, ammonium acetate, acetic acid, sodium acetate, sodium methoxide, sodium ethoxide, and a combination thereof.

In alternative embodiments, the catalyst is glycerol.

In alternative embodiments, the solvent is a primary or a secondary amine. The primary amine can be an aliphatic amine, an alkanolamine, a branched chain amine, an ether amine, a cyclic amine, an aryl amine, or any combination thereof. The amount of primary amine in the reaction mixture is between about 1 mol to about 100 mol per mol of feedstock, or between about 10 mol to about 70 mol per mol of feedstock, or about 30 mol to about 50 mol per mol of feedstock.

In alternative embodiments, the invention provides processes for the production of free fatty acids from a lipid-containing feedstock comprising phospholipids and, optionally, other lipids e.g. glycerides, the process comprising:

(a) providing a reaction mixture that comprises the feedstock, a solvent comprising water, and CO₂, wherein the water and CO₂ react to form carbonic acid; and

(b) reacting the reaction mixture at a temperature and pressure and for a time that is sufficient to provide for the alcoholysis (esterification) and/or acidification of substantially all of the phospholipids and, if present, the other lipids in the feedstock, wherein optionally the time is between about 0 to about 24 hours, between about 0 to about 12 hours, e.g., between about 1 hour and about 8 hours, between about 3 hours and 5 hours, between about 5 minutes and one hour, between about 2 minutes to about 20 minutes, or between about 0 minutes to about 1 minute.

In alternative embodiments, the feedstock comprises from between about 10 wt % to about 100 wt % phospholipids, or from between about 20 wt % to about 100 wt % phospholipids, about 30 wt % to about 90 wt %, about 40 wt % to about 80 wt %, or about 50 wt % to about 75 wt % phospholipids. The feedstock can further comprise between about 5 wt % to about 40 wt % free fatty acids, and between about 5 wt % to about 50 wt % glycerides.

In alternative embodiments, the feedstock is an oil gum derived from a crude vegetable oil or a mixture of oil gums derived from multiple vegetable oils. The crude vegetable oil can be selected from the group consisting of soybean oil, palm oil, canola oil, rapeseed oil, wheat germ oil, safflower oil, peanut oil, cottonseed oil, sunflower oil, grape seed oil, jatropha oil, palm kernel oil, coconut oil, olive oil, corn oil, hazelnut oil, linseed oil, rice bran oil, safflower oil, sesame oil, algae oil, and a combination thereof.

In alternative embodiments, the reaction temperature is between about 10° C. to about 175° C., the pressure of the reaction is between about between about 0 psi and about 2500 psi, or between about 500 psi and about 2000 psi, between about 800 psi and about 1750 psi, or about 1000 psi and about 1500 psi, or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi, and the time of the reaction is between about 0 to about 12 hours.

In alternative embodiments, the amount of water in the reaction mixture is between about 1 mol to about 100 mol per mol of feedstock, or between about 10 mol to about 70 mol per mol of feedstock, or about 30 mol to about 50 mol per mol of feedstock.

In alternative embodiments, the amount of CO₂ in the reaction mixture is between about 1 mol to about 10 mol per mol of feedstock.

In alternative embodiments, the reaction mixture further comprises an alcohol, and wherein in the reaction the alcohol esterifies at least a portion of the lipids in the feedstock to generate fatty alkyl esters. In alternative embodiments, the amount of alcohol in the reaction mixture is between about 1 mol to about 100 mol per mol of feedstock, or between about 10 mol to about 70 mol per mol of feedstock, or about 30 mol to about 50 mol per mol of feedstock. The alcohol can contain between 1 and 9 carbon atoms, or the alcohol is a methanol, an ethanol, a butanol, an isopropyl alcohol, a sec-butanol, a t-butanol, a benzyl alcohol, or a combination thereof.

In alternative embodiments, the invention provides methods and processes for the production of fatty amides from lipid feedstocks comprising phospholipids and, optionally, other lipids, e.g. glycerides, the process comprising:

(a) providing a reaction mixture that comprises the feedstock, a solvent comprising an ammonia, and a catalyst; and

(b) reacting the reaction mixture at a temperature and pressure and for a time that is sufficient to provide for the solvolysis of substantially all of the phospholipids and, if present, the other lipids in the feedstock,

wherein optionally the time is between about 0 to about 24 hours, between about 0 to about 12 hours, e.g. between about 1 hour and about 8 hours, between about 3 hours and 5 hours, between about 5 minutes and one hour, between about 2 minutes to about 20 minutes, or between about 0 minutes to about 1 minute.

In alternative embodiments, the feedstock comprises from between about 10 wt % to about 100 wt % phospholipids, or from between about 20 wt % to about 100 wt % phospholipids, about 30 wt % to about 90 wt %, about 40 wt % to about 80 wt %, or about 50 wt % to about 75 wt % phospholipids. The feedstock can further comprise between about 5 wt % to about 40 wt % free fatty acids, and between about 5 wt % to about 50 wt % glycerides.

In alternative embodiments, the feedstock is an oil gum derived from a crude vegetable oil. The gum can be derived from a crude vegetable oil selected from the group consisting of a soybean oil, palm oil, canola oil, rapeseed oil, wheat germ oil, safflower oil, peanut oil, cottonseed oil, sunflower oil, algae oil, or a combination thereof.

In alternative embodiments, the reaction temperature is between about 10° C. to about 175° C., the pressure of the reaction is between about 0 psi and about 2500 psi, or between about 500 psi and about 2000 psi, between about 800 psi and about 1750 psi, or about 1000 psi and about 1500 psi, or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi, and the time of the reaction is between about 0 to about 12 hours.

In alternative embodiments, the amount of ammonia in the reaction mixture is between about 1 mol to about 100 mol per mol of feedstock, or between about 10 mol to about 70 mol per mol of feedstock, or about 30 mol to about 50 mol per mol of feedstock.

In alternative embodiments, the catalyst is an ammonium salt; or, the catalyst is selected from the group consisting of ammonium nitrate, ammonium acetate, acetic acid, sodium acetate, sodium methoxide, sodium ethoxide and a combination thereof.

In alternative embodiments, the catalyst is glycerol.

In alternative embodiments, the invention provides methods and processes for the production of fatty amides from lipid feedstocks comprising phospholipids and, optionally, other lipids, e.g. glycerides, the process comprising:

(a) providing a reaction mixture that comprises the feedstock and a solvent comprising a primary amine or a secondary amine, and optionally, a catalyst; and

(b) reacting the reaction mixture at a temperature and pressure and for a time that is sufficient to provide for the solvolysis of substantially all of the phospholipids and, if present, the other lipids in the feedstock,

wherein optionally the time is between about 0 to about 24 hours, between about 0 to about 12 hours, e.g. between about 1 hour and about 8 hours, between about 3 hours and 5 hours, between about 5 minutes and one hour, between about 2 minutes to about 20 minutes, or between about 0 minutes to about 1 minute.

In alternative embodiments, the feedstock comprises between about 10 wt % to about 100 wt % phospholipids; or, the feedstock further comprises between about 5 wt % to about 40 wt % free fatty acids, and between about 5 wt % to about 50 wt % glycerides.

In alternative embodiments, the feedstock is an oil gum derived from a crude vegetable oil; or, the gum is derived from a crude vegetable oil selected from the group consisting of soybean oil, palm oil, canola oil, rapeseed oil, wheat germ oil, safflower oil, peanut oil, cottonseed oil, sunflower oil and algae oil.

In alternative embodiments, the reaction temperature is between about 10° C. to about 175° C., the pressure of the reaction is between about 0 psi and about 2500 psi, or between about 500 psi and about 2000 psi, between about 800 psi and about 1750 psi, or about 1000 psi and about 1500 psi, or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi, and the time of the reaction is between about 0 to about 12 hours.

In alternative embodiments, the amount of primary amine in the reaction mixture is between about 1 mol to about 100 mol per mol of feedstock, or between about 10 mol to about 70 mol per mol of feedstock, or about 30 mol to about 50 mol per mol of feedstock.

In alternative embodiments, the amount of catalyst in the reaction mixture is between about 1 mol to about 10 mol per mol of feedstock.

In alternative embodiments, the catalyst is an ammonium salt; or, the catalyst is selected from the group consisting of ammonium nitrate, ammonium acetate, acetic acid, sodium acetate, sodium methoxide, sodium ethoxide and a combination thereof.

In alternative embodiments, the catalyst comprises a glycerol.

In alternative embodiments, the primary amine is an aliphatic amine, an alkanolamine, a branched chain amine, an ether amine, a cyclic amine, an aryl amine, or any combination thereof.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

FIG. 1 is a schematic diagram of an exemplary method of producing free fatty acids and other products from feedstocks comprising phospholipids, as described in detail, below.

FIG. 2 is a schematic diagram of an exemplary method of producing free fatty acids, fatty alkyl esters and other products from feedstocks comprising phospholipids using water as a solvent, as described in detail, below.

FIG. 3 is a schematic diagram of an exemplary method of producing amides and other products from feedstocks comprising phospholipids using water and alcohol as solvents, as described in detail, below.

FIG. 4 is a schematic diagram of an exemplary method of producing amides and other products from feedstocks comprising phospholipids using ammonia as a solvent, as described in detail, below.

FIG. 5 is a schematic diagram of an exemplary method of producing amides and other products from feedstocks comprising phospholipids using primary or secondary amines, as described in detail, below.

Reference will now be made in detail to various exemplary embodiments of the invention. The following detailed description is provided to give the reader a better understanding of certain details of aspects and embodiments of the invention, and should not be interpreted as a limitation on the scope of the invention.

### DETAILED DESCRIPTION OF THE INVENTION

In alternative embodiments, the present invention provides methods and industrial processes for the economically efficient conversion or processing of phospholipid-comprising feedstocks, e.g., low-value feedstocks such as phospholipid-comprising oil gums, including oil gums generated during the degumming process of vegetable oil refining, e.g. oil gums, to free fatty acids, fatty alkyl esters, glycerol, derivatives, e.g. glycerol choline phosphatase, fatty amides, fatty amines or related compounds, or any combination thereof. In alternative embodiments, the phospholipids in the feedstocks comprise phosphatidylcholine, phosphatidylethanolamine, phosphatidylinositol, phosphatidic acid, or related phospholipids. In alternative embodiments, the invention involves performing acidification and/or solvolysis, e.g., hydrolysis, alcoholysis (esterification), ammonolysis, or a combination thereof, on a feedstock material comprising phospholipids.

In alternative embodiments, the phospholipid-comprising feedstock can be or comprise a waste stream produced during the degumming of vegetable oils such as wheat germ oil, corn oil, soybean oil, safflower oil, peanut oil, cottonseed oil, sunflower oil, rapeseed oil, palm oil, canola oil, algae oil; or, any other animal or vegetable oil, or any combination thereof.

In alternative embodiments, the invention provides methods and industrial processes comprising reacting a phospholipid-comprising feedstock with water and an alcohol, and optionally a carbon dioxide, to make a “starting mixture; then, subjecting the starting mixture to a temperature of about 150° C. to about 300° C., and a pressure of about 1500 psi to about 2500 psi. In alternative embodiments, the reaction time is in the range of between about 0 to about 24 hours, between about 0 to about 12 hours, e.g. between about 1 hour and about 8 hours, between about 3 hours and 5 hours, between about 2 minutes to about 20 minutes, or between about 0 minutes to about 1 minute, or between about 5 minutes to about 120 minutes. In a conventional stirred tank reactor, the reaction time can be in the range of between about 60 to 90 minutes for a batch reactor. For any particular reaction, at higher temperatures, the reaction times can be correspondingly reduced.

In alternative embodiments, the invention provides methods and industrial processes comprising reacting a phospholipid-comprising feedstock with water, and optionally a carbon dioxide, to make a “first starting mixture” (also called a “slurry mixture”; then, subjecting the first starting mixture to a temperature of about 150° C. to about 300° C., and a pressure of about 1500 psi to about 2500 psi. In alternative embodiments, the reaction time is in the range of between about 0 to about 24 hours, between about 0 to about 12 hours, e.g. between about 1 hour and about 8 hours, between about 3 hours and 5 hours, between about 2 minutes to about 20 minutes, between about 5 minutes and one hour, or between about 0 minutes to about 1 minute.

This reaction of the “first starting mixture” produces a “product mixture”, or a “second mixture”, and in alternative embodiments, the second mixture is mixed with alcohol to produce a “third mixture”, which is reacted or subjected to a temperature of between about 150° C. to about 300° C., and a pressure of between about 1500 psi to about 2500 psi. In alternative embodiments, the reaction time is in the range of between about 0 to about 12 hours, e.g. between about 1 hour and about 8 hours, between about 3 hours and 5 hours, between about 2 minutes to about 20 minutes, or between about 0 minutes to about 1 minute.

In alternative embodiments, the pH of the reaction, or any step of the complete reaction process (e.g., reacting the first starting mixture, versus reacting the third mixture), can be anywhere between about pH 0 and pH 7.4, or between about pH 0.5 and pH 7.0, or between about 0 and pH 7, or about pH 1, 1.5, 2, 2.5, 3, 3, 5, 4, 4, 5, 5, 5.5 6, 6.5, 7, 7.5 or more (or more basic).

In alternative embodiments, the temperature of the reaction, or any step of the complete reaction process (e.g., reacting the first starting mixture, versus reacting the third mixture), can be anywhere between about 100° C. to about 350° C., or between about 150° C. to about 300° C.

In alternative embodiments, the pressure of the reaction, or any step of the complete reaction process (e.g., reacting the first starting mixture, versus reacting the third mixture), can be anywhere between about 1000 psi to 3000 psi, or between about 1500 psi to about 2500 psi, or the pressure of the reaction operation can be slightly in excess of vapor pressure of the alcohol used in the reaction (e.g., a methanol) at the selected operating temperature, for example at a pressure of about 10, 15, 20, or 25 or more psi over the vapor pressure of the alcohol.

In alternative embodiments, the slurry mixture is subjected to any of these exemplary reaction conditions, and if present, the carbon dioxide reacts with water to form carbonic acid. Acidification of reaction conditions by carbonic acid accelerates hydrolysis of the fatty acids from the phospholipids, resulting in the formation of free fatty acids and phosphate. The alcohol present in the reaction then esterifies the free fatty acids and alkylates them to form fatty acid esters. When, in alternative embodiments, the alcohol is methanol, the free fatty acids liberated from the soaps would react with the methanol to form fatty acid methyl esters (FAME), which can be used as biodiesel, and has been referred to as biodiesel. Mono-, di-, and triglycerides present in the feedstock are hydrolyzed under any of these exemplary reaction conditions, and result is the separation of fatty acids from the glycerin group. In alternative embodiments, the fatty acids are then esterified to form fatty acid esters (such as FAMEs, if methanol is used as one of the alcohols). Any acyl-glycerol (e.g., mono-, di-, and tri-glycerides) will then react with the alcohol to form a glycerol and fatty acids.

In alternative embodiments, the reaction is carried out as a two phase process, wherein the first phase comprises a first solvolysis (e.g., an alcoholysis or ammonolysis stage) uses water, ammonia or a primary or a secondary amine as a solvent, and optionally carbon dioxide, and then in a second phase alcohol is used, e.g., the second phase comprises an esterification and an alkylation stage using alcohol. In alternative embodiments, in the first phase, e.g., where the first phase is a hydrolysis stage, water and carbon dioxide are mixed with the gum feedstock and placed in a reactor. During this exemplary phase, the carbon dioxide reacts with the water to form carbonic acid and any bound fatty acids, e.g., phospholipids and glycerides (acylglycerols), and are hydrolyzed to form a reaction slurry of free fatty acids, phosphate and glycerol (also called glycerine or glycerin).

In alternative embodiments, during the second esterification and alkylation phase, the reaction slurry from the first hydrolysis phase is fed back into the reactor where it is mixed with an alcohol. During the second esterification and alkylation phase, free fatty acids (FFAs) produced during the hydrolysis phase react with the alcohol resulting in the esterification and alkylation of the FFAs and forming fatty acid esters, e.g., FAME, or biodiesel.

In alternative embodiments, the invention provides methods and industrial processes for the economically efficient conversion of feedstocks comprising phospholipids, e.g., phospholipid-comprising oil gums, into high-value, renewable fuels, e.g., biodiesel fuel, and chemicals including free fatty acids, fatty alkyl esters, fatty amides, fatty amines, or a combination thereof.

In alternative embodiments, the invention provides fatty acids and fatty acid derivatives, for use in a variety of commercial applications. Derivatives of fatty acids include, but are not limited to fatty alkyl esters e.g. fatty acid methyl esters, or FAMEs (which can be used as biodiesel fuel), fatty amides, and fatty amines.

By generating fatty acids from phospholipids without the use of expensive solvents or catalysts, and without relying on extreme temperatures and pressures, exemplary processes of this invention can enable the conversion of waste or low-value products (e.g., “waste streams”) comprising phospholipids into higher value chemicals and intermediates. Once separated from their glycerol-phosphate structures, fatty acids, e.g., FAMEs, can be converted into a variety of high-value chemicals and/or fuels.

The phospholipid-comprising feedstock used in the various embodiments can be, for example, any phospholipid-comprising oil gum, including oil gums generated during the degumming process of vegetable oil refining, herein referred to as “gums”. The degumming process is used primarily to remove phospholipids from natural oils, such as vegetable oil. Gums that can be used as a feedstock for methods of the invention can comprise any phospholipid, along with other lipids and lipid derivatives. The gum feedstock can be derived from any natural oil, for example, wheat germ oil, corn oil, soybean oil, safflower oil, peanut oil, cottonseed oil, sunflower oil, rapeseed oil, palm oil, canola oil, algae oil, or any other animal or vegetable oil. In certain embodiments, the feedstock can be, for example, a refined or semi-refined gum product such as lecithin (which can be a mixture of phosphoric acids, choline, fatty acids, glycerol, glycolipids, triglycerides, and phospholipids).

The feedstock can contain from about 10 wt % to about 100 wt % phospholipids, e.g. from about 20 wt % to about 100 wt % phospholipids, about 30 wt % to about 90 wt %, about 40 wt % to about 80 wt %, or about 50 wt % to about 75 wt % phospholipids. The feedstock can contain from about 1 wt % to about 40 wt % free fatty acids (FFA), e.g. about 5 wt % to about 20 wt % FFA. The feedstock can contain from about 5 wt % to about 50 wt % glycerides e.g. from about 20 wt % to about 40 wt % glycerides. The forgoing amounts are exemplary and do not restrict the composition of the feedstock that can be used in the methods of any embodiments of present invention. Those skilled in the art will recognize that the composition of oil gums can vary significantly without limiting the methods of the present invention and may include other products in varying amounts, including, without limitation, sterols, carbohydrates, or the like. Each of the amounts for the components listed above is based on the dry weight of the feedstock.

In alternative embodiments, the feedstock is substantially free of water, or has been processed to be substantially free of water.

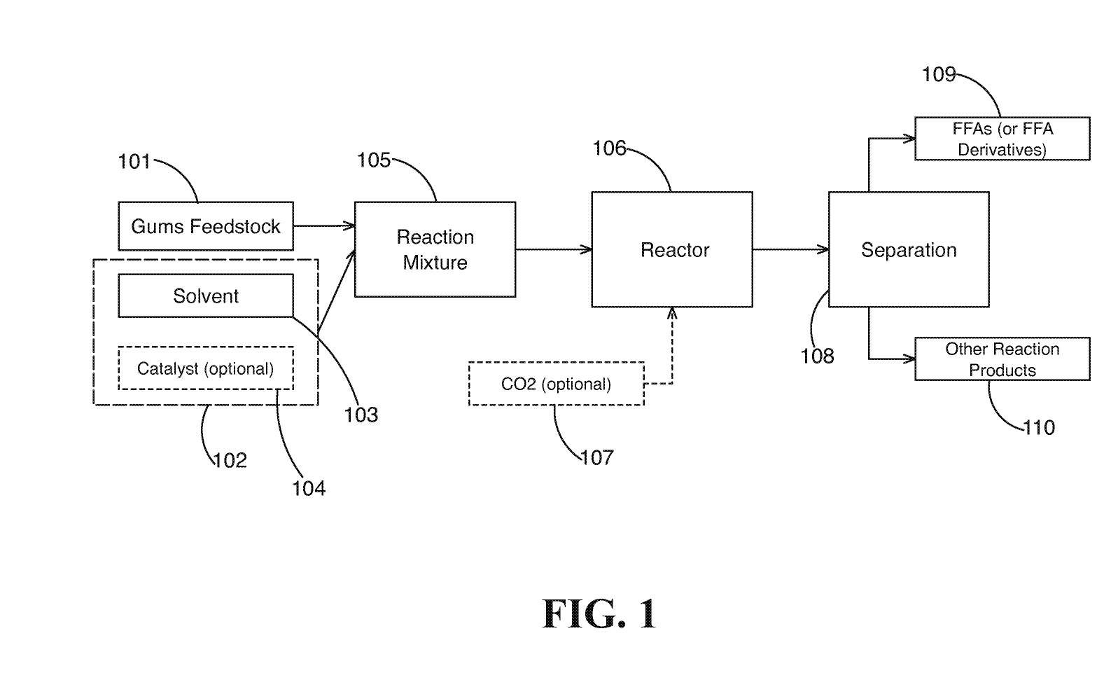

Referring now to FIG. 1, which schematically illustrates an exemplary method of the invention for producing free fatty acids and other products from feedstocks: prior to entering the reactor 106, the feedstock 101 is combined with a fluid (which acts as a solvent 103, which in alternative embodiments can be only the solvent (e.g., water), or solvent and a catalyst 104) 102 to form a mixture 105. In alternative embodiments, the fluid 102 is comprised of a solvent 103 and, optionally, a catalyst 104. The mixture 105 is then fed to the reactor 106 wherein the heat and pressure are sufficient to facilitate one or more reactions that result in the generation of free fatty acids from the phospholipids. Optionally, CO₂ 107 is fed to the reactor wherein it reacts with water (if present in the reaction mixture) to form carbonic acid, which serves to acidify some amount of the esters present in the feedstock. In alternative embodiments, following the reaction, the reaction slurry (or “slurry mixture”) can be selectively separated 108 into discreet products 109, 110. These products can include, for example, free fatty acids, fatty alkyl esters, glycerol, glycerol derivatives, or a combination thereof. The composition of the reaction slurry will vary depending on the fluid used (for example, what solvent was used), or whether a catalyst was used or not, in a particular embodiment, as well as the corresponding reaction conditions.

In any of the embodiments, the reactor system can be batch or continuous. In alternative embodiments, any conventional pressure vessel systems can be used to practice this invention, including reactors that will operate in batch and continuous modes. In alternative embodiments, a continuous pipe-type, e.g. a plug-flow reactor can be used to carry out the reactions. In alternative embodiments, the reactor is a pipe with sufficient residence time to allow for the reaction to complete and is operated under target pressure and temperature range. The pipe can allow for reasonable reaction to occur with minimized vessel complexity.

In certain embodiments of methods and processes of the invention, the fluid, or solvent, comprises water. In other embodiments, the fluid, or solvent, comprises water and an alcohol. In further embodiments, the fluid, or solvent, comprises an ammonia derivative, e.g. a primary amine or a secondary amine. In still further embodiments, the feedstock is not mixed with a fluid, or solvent, prior to entering the reactor.

The alcohol used in the various embodiments can be, for example, methanol, ethanol, propanol, butanol, isopropyl alcohol, sec-butanol, t-butanol, benzyl alcohol, or a combination thereof. In alternative embodiments, alcohols containing 1 to 5, 6, 7, 8, 9 or more carbons are used. However, there may be specific situations and conditions wherein higher alcohols could be used. For purposes of this discussion, methanol is used as the alcohol; however, those skilled in the art would understand that other alcohols may be used.

In certain embodiments, CO₂ is fed into the reactor in order to facilitate the acidification of the phospholipids into free fatty acids and glycerol derivatives. In such embodiments, the CO₂ reacts with the water (acting as the solvent) to form carbonic acid. The resulting carbonic acid serves to acidify all or a portion of the phospholipids and, as a result, lower temperatures and pressure are required in the reaction vessel for a favorable conversion of phospholipids into free fatty acids and glycerol derivatives.

Phospholipid Hydrolysis with Water and CO₂:

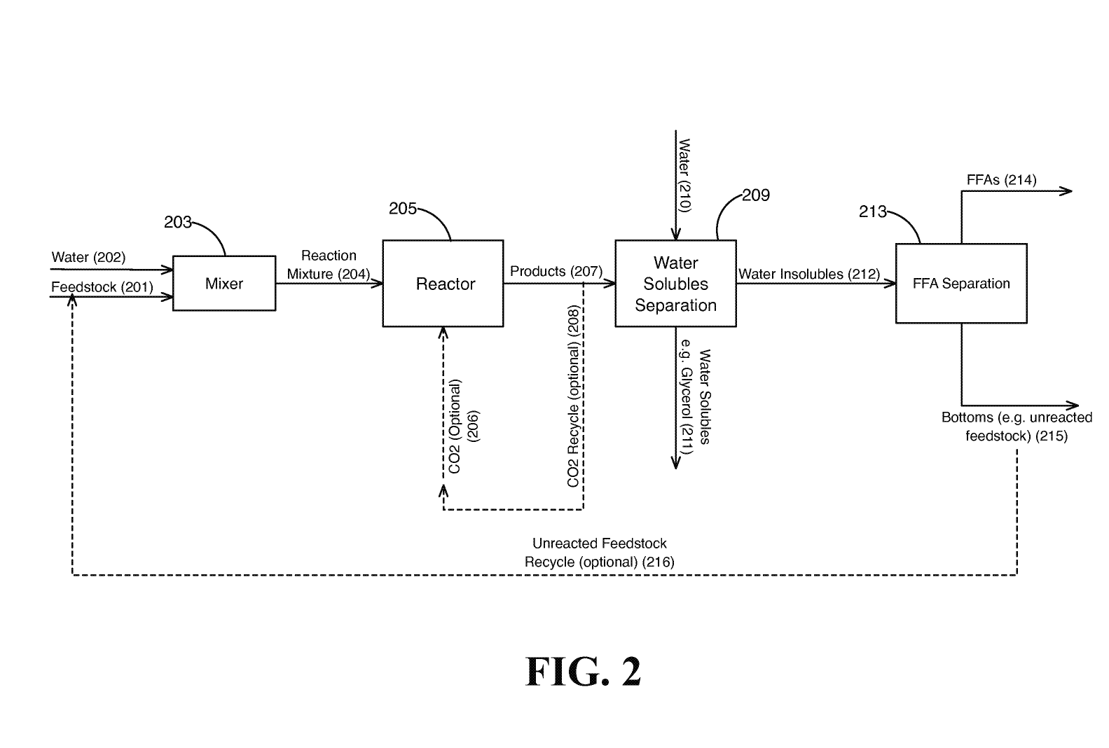

Referring to the exemplary method as set forth in FIG. 2, an exemplary process of the invention for producing free fatty acids, fatty alkyl esters and other products from feedstocks comprising phospholipids, certain embodiments provide for the generation of free fatty acids from a feedstock comprising phospholipids initiated by mixing the feedstock 201 with water (as the solvent) 202 using any suitable mixer 203 and feeding the mixture 204 to a reactor 205 wherein it is subjected to temperatures and pressures sufficient to cleave, via hydrolysis, the fatty acids from the phospholipids. Optionally, CO₂ is fed to the reactor 206 wherein it reacts with the water to form carbonic acid, thereby facilitating the generation of free fatty acids by acidifying all or a portion of the phospholipids. If present in the feedstock, all or a portion of the glycerides (acylglycerols) can be subjected to hydrolysis and/or acidification in the same reaction step, thereby generating free fatty acids and glycerol from the glycerides.

The amount of water (as the solvent) in the mixture can be in the range of between about 1 mol to about 100 mol per mol of feedstock, e.g. between about 10 mol to about 70 mol per mol of feedstock, or about 30 mol to about 50 mol per mol of feedstock. The amount of CO₂ added to the reactor can be in the range of about 1 mol to about 10 mol per mol of feedstock, e.g. between about 3 mol to about 7 mol per mol of feedstock.

The temperature of the reactor can be in the range of between about 10° C. to about 175° C., e.g. between about 40° C. and about 120° C. or between about 75° C. and about 90° C. The pressure of the reactor can be in the range of between about 0 psi and about 2500 psi, e.g. between about 500 psi and about 2000 psi, between about 800 psi and about 1750 psi, or about 1000 psi and about 1500 psi. The reaction time can be in the range of between about 0 to about 12 hours, e.g. between about 1 hour and about 8 hours, between about 3 hours and 5 hours, between about 2 minutes to about 20 minutes, or between about 0 minutes to about 1 minute.

For example, in an exemplary solvolysis (i.e. hydrolysis) reaction, where the solvent is water, without CO₂, the amount of feedstock used is between about 5-10% of the total weight of the reaction mixture, the amount of water used is 90-95% of the total weight of the reaction mixture, the temperature is between about 120-140° C., the pressure is between about 500-1500 psig, and the reaction time is between about 1-12 hr.

For example, in an exemplary solvolysis (i.e. hydrolysis) reaction, where the solvent is water, with CO₂, the amount of feedstock used is between about 5-10% of the total weight of the reaction mixture, the amount of water used is 90-94% of the total weight of the reaction mixture, the amount of CO₂ used is 0.01-1% of the total weight of the reaction mixture, the temperature is between about 120-140° C., the pressure is between about 500-1500 psig, and the reaction time is between about 1-12 hr.

Following the reaction, the reaction slurry can comprise a variety of products 207, e.g. free fatty acids, glycerol, glycerol derivatives, e.g. glycerol phosphatidylcholine and/or other glycerol phosphatidyls, unreacted glycerides, or a combination thereof. The products can then be selectively removed from the reaction slurry and separated into discreet fractions. These fractions may then be sold as end products or used as intermediates to be upgraded into value-added products in downstream processes.

Following the reaction, un-reacted CO₂ can be separated from the reaction slurry and recycled for use in subsequent reactions 208. Several techniques are suitable for this step and it would be understood by those in the art that any of these techniques may be applied.

In alternative embodiments comprising the addition of CO₂ in the reaction, un-reacted CO₂ can be separated from the reaction slurry, for example, using a flash separation technique wherein the slurry mixture is transferred to a flash drum or appropriate or equivalent vessel wherein the pressure is reduced from the pressure within the reactor, wherein the pressure in the reactor is above the vapor pressure of the CO₂ for the corresponding reaction temperature, to a pressure of about atmospheric pressure. The decrease in pressure results in an environment in which the vapor pressure of the CO₂ exceeds its external pressure (the pressure of the flash drum of vessel), allowing for the CO₂ to vaporize or “flash” out of the product slurry.

In certain embodiments, the reaction slurry can be fed to a water-solubles separation unit 209 following the reaction wherein the water-soluble products 211 (such as glycerol or glycerol phosphatidyls) are removed from the slurry. The method used in the water-solubles separation step can be any of several methods known in the art, e.g. a water wash or a counter-current water wash wherein the reaction slurry is contacted with water 210 to generate a water phase comprising those products in the reaction slurry that are water soluble. Those skilled in the art would recognize that other separation methods would be suitable for this step. Water-soluble products can include, for example, glycerol and/or glycerol derivatives e.g. glycerol phosphatidyls.

After the water-soluble products are separated, the remaining water-insoluble products 212 can be separated into discreet fractions, e.g., free fatty acids 214 and unreacted glycerides or any unreacted feedstock or component of the feedstock that did not undergo hydrolysis or acidification 215. The method for this step can be any of several known in the art, for example distillation, crystallization or the like. Any unreacted feedstock 215 can optionally be recycled for use in subsequent reactions.

Phospholipid Hydrolysis/Alcoholysis with Water, Alcohol, and Optionally CO₂:

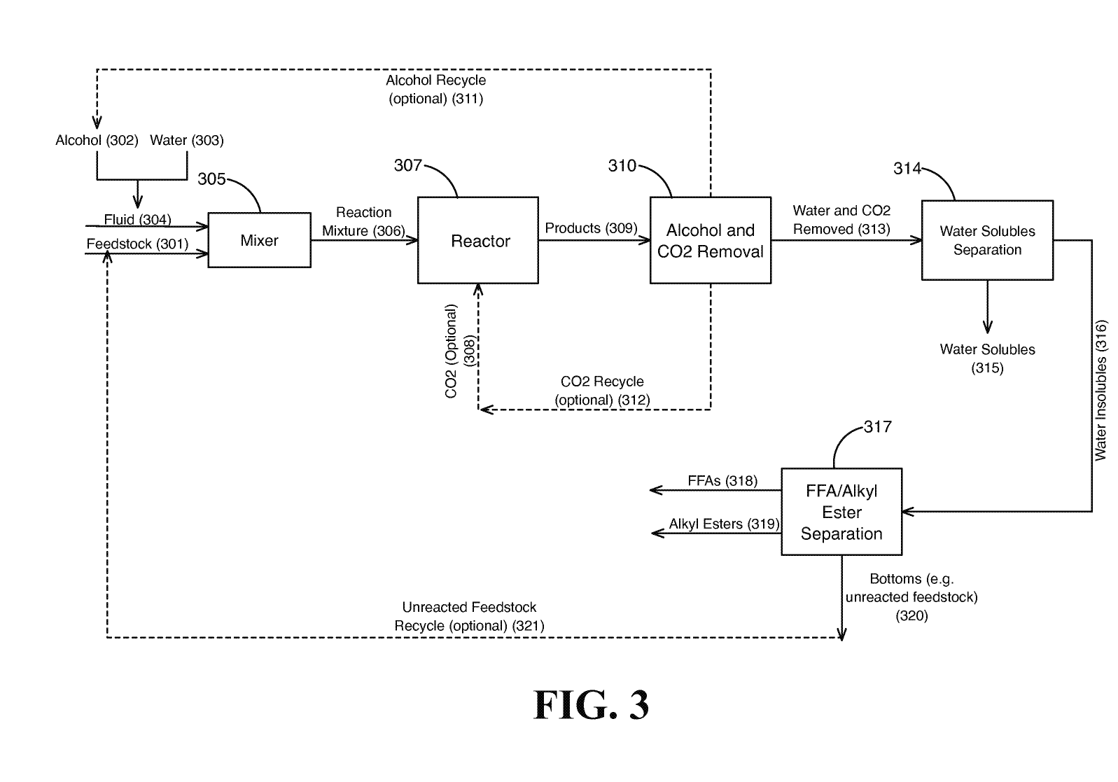

Referring to the exemplary method as set forth in FIG. 3, an exemplary method of the invention for producing amides and other products from feedstocks comprising phospholipids, certain embodiments provide for the generation of free fatty acids, alkyl esters, or a combination thereof from a feedstock comprising phospholipids, initiated by mixing the feedstock 301 with a fluid (i.e., the solvent) 304 comprising water 303 and alcohol 302 using any suitable mixer 305, and feeding the mixture 306 to a reactor 307 wherein it is subjected to temperatures and pressures sufficient to generate fatty acids via hydrolysis and/or alcoholysis from the phospholipids. Optionally, CO₂ is fed 308 to the reactor 307 wherein it reacts with the water to form carbonic acid, thereby facilitating the generation of free fatty acids by acidifying a portion of the phospholipids. If present in the feedstock, all or a portion of the glycerides can be subjected to hydrolysis and/or alcoholysis (i.e. esterification), and/or acidification in the same reaction step, thereby separating the fatty acids from the glycerides and generating glycerol and free fatty acids.

The amount of water (as the solvent) in the mixture can be in the range of between about 1 mol to about 100 mol per mol of feedstock, e.g. between about 10 mol to about 70 mol per mol of feedstock, or about 30 mol to about 50 mol per mol of feedstock. The amount of alcohol in the mixture can be in the range of between about 1 mol to about 100 mol per mol of feedstock, e.g. between about 20 mol to about 80 mol per mol of feedstock, or about 40 mol to about 60 mol per mol of feedstock. The amount of CO₂ added to the reactor can be in the range of about 1 mol to about 100 mol per mol of feedstock, e.g. between about 30 mol to about 70 mol per mol of feedstock.

The temperature of the reactor can be in the range of between about 10° C. to about 175° C., e.g. between about 40° C. and about 120° C. or between about 75° C. and about 90° C. The pressure of the reactor can be in the range of between about 0 psi and about 2500 psi, e.g. between about 500 psi and about 2000 psi, between about 800 psi and about 1750 psi, or about 1000 psi and about 1500 psi. The reaction time can be in the range of between about 0 to about 12 hours, e.g. between about 1 hour and about 8 hours, between about 3 hours and 5 hours, between about 2 minutes to about 20 minutes, or between about 0 minutes to about 1 minute.

For example, in an exemplary alcoholysis (i.e. esterification) reaction, where the solvent is water and alcohol, with CO₂, the amount of feedstock used is between about 5-10% of the weight of the total weight of the reaction mixture, the amount of water used is between about 5-50% by weight of the total weight of the reaction mixture, the amount of alcohol used is between about 5-50% by weight of the total weight of the reaction mixture, the amount of CO₂ used is 0.01-1% of the weight of the total weight of the reaction mixture, the temperature is between about 120-140° C., the pressure is between about 500-1500 psig, and the reaction time is between about 1-12 hours.

When alcohol and water are present in the fluid (i.e., the solvent), the reaction conditions provide for the simultaneous hydrolysis and/or alcoholysis (i.e. esterification) of phospholipids to generate some combination of free fatty acids and fatty alkyl esters in the reaction product, or “reaction slurry” 309. Those phospholipids that are subjected to hydrolysis only will generate free fatty acids and glycerol-phosphatidyls. Those phospholipids that are subjected to esterification or transesterification will generate fatty alkyl esters, e.g. fatty acid methyl esters (FAME) if methanol is the selected alcohol in the embodiment, and glycerol-phosphatidyls.

Similarly, if glycerides are present in the feedstock, they will be subjected to simultaneous hydrolysis and/or esterification or transesterification to generate glycerol, free fatty acids, fatty alkyl esters, or a combination thereof. The addition of alcohol in the fluid (i.e., the solvent) can allow for lower temperatures and pressures in the reactor to achieve favorable generation of free fatty acids and alkyl esters due to alcohols increased solvolysis activity.

Following the reaction, un-reacted CO₂ can be separated from the reaction slurry and recycled 312 for use in subsequent reactions. Several techniques are suitable for this step and is would be understood by those in the art that any of these techniques may be applied. In alternative embodiments comprising the addition of CO₂ in the reaction, un-reacted CO₂ can be separated from the reaction slurry, for example, using a flash separation technique wherein the slurry mixture is transferred to a flash drum or appropriate or equivalent vessel wherein the pressure is reduced from the pressure within the reactor, wherein the pressure in the reactor is above the vapor pressure of the CO₂ for the corresponding reaction temperature, to a pressure of about atmospheric pressure. The decrease in pressure results in an environment in which the vapor pressure of the CO₂ exceeds its external pressure (the pressure of the flash drum of vessel), allowing for the CO₂ to vaporize or “flash” out of the product slurry. The recovered CO₂ can then be recovered for use in the process during subsequent reactions.

Similarly, un-reacted alcohol (and carbon dioxide) can be separated 310 from the reaction slurry at this stage and, optionally, recycled for subsequent reactions 311 (un-reacted alcohol) or 312 (carbon dioxide). The alcohol separation may be achieved by any of several known methods in the art, for example a flash separation. In alternative embodiments, the product slurry undergoes a flash process wherein the product mixture is transferred to a flash drum or appropriate or equivalent vessel wherein the pressure is reduced to from the pressure within the reactor, to, for example, about atmospheric pressure, or about less than 14 psi, e.g. less than 1 psi, or about 0.1 psi. The decrease in pressure results in an environment in which the vapor pressure of the alcohol, e.g., methanol, exceeds its external pressure (the pressure of the flash drum or vessel), allowing for the alcohol, e.g., methanol, and water (collectively referred to as “the solvent” in this and subsequent steps) to vaporize or “flash” out of the product mixture. The recovered alcohol can be recycled for use in the process during subsequent reactions.

In alternative embodiments, the CO₂ and the alcohol are recovered in the same separation step, e.g. a flash separation step.

In certain embodiments, the reaction slurry 313 (wherein the alcohol and CO₂ have been removed 310) can be fed to a water-solubles separation 314 unit, where water-soluble products 315 are removed from the slurry 313. The method used in the water-solubles separation step can be any of several methods known in the art, e.g. a water wash. Those skilled in the art would recognize that other separation methods would be suitable for this step. Water-soluble products can include, for example, glycerol and glycerol derivatives e.g. glycerol phosphatidyls.

In alternative embodiments, the reaction slurry (wherein the alcohol and CO₂ have been removed) is transferred to a static mixer wherein it is mixed with water. The water and reaction slurry mixture is then transferred to a decanter wherein an oil (lipid) stream, comprising any free fatty acids as well as esters, e.g. fatty acid alkyl esters and any unreacted feedstock, and an aqueous stream are formed and are separated.

The aqueous stream (comprising glycerol and any derivatives thereof, any of the alcohol, e.g., methanol that was not removed in the previous alcohol separation step, and water) is then transferred to a glycerol stripping column, e.g. a 4-stage stripping column, in which the aqueous stream is introduced to the top of the column and, upon contacting the bottom of the column is heated such that a vapor phase, comprising primarily the alcohol e.g. methanol and water, is generated and rises to the top of the column where it is removed. In this exemplary embodiment, the column “bottoms” are a primarily a glycerol product that can be, for example, in the range about 80 to 88 wt % glycerol, e.g. about 85% glycerol.

In alternative embodiments, the alcohol (e.g. methanol)/water product is sent to the alcohol recovery unit wherein it is distilled to yield a substantially pure alcohol, e.g., methanol, product.

After the water-soluble products are separated, the remaining water-insoluble products 316 (e.g., a lipid stream resulting from the water-washing step) can be separated into discreet fractions 317, e.g., free fatty acids 318, fatty alkyl esters 319 and glycerides or any unreacted feedstock 320. The method for this step can be any of several known in the art, for example distillation, crystallization or the like. Any unreacted feedstock can optionally be recycled 321 for use in subsequent reactions.

Ammonolysis of Phospholipids:

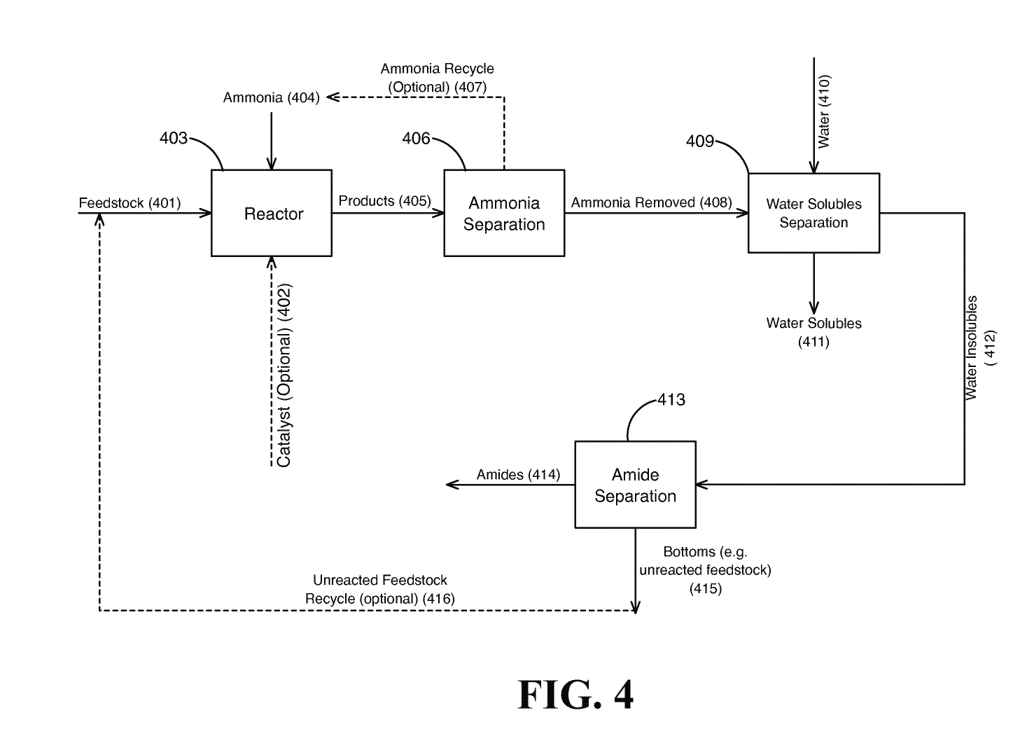

Referring to the exemplary method as set forth in FIG. 4, an exemplary method of the invention for producing amides and other products from feedstocks 401 comprising phospholipids, certain embodiments provide for the generation of fatty amides from a feedstock 401 comprising phospholipids initiated by optionally mixing the feedstock 401 with a catalyst 402, e.g. an ammonium salt catalyst such as ammonium chloride, and feeding the mixture (if the catalyst is present) or the feedstock to a reactor 403 wherein it is contacted with ammonia (as the solvent) 404 and subjected to temperatures and pressures sufficient to allow for ammonolysis, thereby cleaving the fatty acids from the phospholipids and generating a product slurry 405. The ammonolysis is achieved by simultaneously feeding ammonia, e.g. gaseous ammonia, to the reactor wherein it reacts with the phospholipids in the presence of the catalyst to generate fatty amides and glycerol-phosphatidyls. If present in the feedstock, all or a portion of the glycerides can be subjected to ammonolysis in the same reaction step, thereby cleaving the fatty acids from the glycerides and generating glycerol and fatty amides.

The catalyst can be, for example, an ammonium salt such as ammonium nitrate or ammonium acetate. Other suitable catalyst include, without limitation, acetic acid, sodium acetate, sodium methoxide, sodium ethoxide, or glycerol.

In alternative embodiments, the ammonolysis process does not include the use of a catalyst.

The amount of catalyst in the reaction mixture can be in the range of between about 0 to about 10 mol per mol of feedstock, e.g. between about 0 mol to about 8 mol, about 0 mol to about 6 mol per mol of feedstock, about 0 mol to about 4 mol of feedstock, or about 0 mol to about 2 mol per mol of feedstock. The amount of ammonia in the reactor can be in the range of between about 1 to about 100 per mol of feedstock, e.g. between about 5 mol to about 85 mol per mol of feedstock, between about 10 mol and about 70 mol per mol of feedstock, between about 15 mol and about 65 mol per mol of feedstock, between about 20 mol to about 50 mol per mol of feedstock, or about 25 mol to about 35 mol of feedstock.

The temperature of the reactor can be in the range of between about 10° C. to about 175° C., e.g. between about 40° C. and about 120° C. or between about 75° C. and about 90° C. The pressure of the reactor can be in the range of between about 0 psi and about 2500 psi, e.g. between about 500 psi and about 2000 psi, between about 800 psi and about 1750 psi, or about 1000 psi and about 1500 psi. The reaction time can be in the range of between about 0 to about 12 hours, e.g. between about 30 minutes and about 10 hours, between about 60 minutes and about 8 hours, between about 90 minutes and about 6 hours, between about 2 hours and 4 hours, or about 2½ hours and 3 hours.

For example, in an exemplary ammonolysis reaction, where the solvent is ammonia, and a catalyst is used, the amount of feedstock used is between about 5-50% of the total weight of the reaction mixture, the amount of ammonia used is between about 5-50% of the total weight of the reaction mixture, the amount of catalyst used is between about 0.01-5% of the total weight of the reaction mixture, the temperature is between about 120-140° C. of the total weight of the reaction mixture, the pressure is between about 1000-1500 psig, and the reaction time is between about 1-12 hours.

Following the reaction, in certain embodiments, un-reacted ammonia and/or catalyst can be separated from the reaction slurry in a suitable device or system 406 and recycled 407 for use in subsequent reactions 403. Several techniques are suitable for this step and it would be understood by those in the art that any of these techniques can be applied. In alternative embodiments, the product slurry undergoes a flash process wherein the product mixture is transferred to a flash drum or appropriate or equivalent vessel wherein the pressure is reduced to from the pressure within the reactor, to, for example, about atmospheric pressure, or about less than 14 psi, e.g. less than 1 psi, or about 0.1 psi. The decrease in pressure results in an environment in which the vapor pressure of the ammonia exceeds its external pressure (the pressure of the flash drum or vessel), allowing ammonia to vaporize or “flash” out of the product mixture. The recovered ammonia can be recycled for use in the process during subsequent reactions.

In certain embodiments, the reaction slurry with optionally the ammonia and/or catalyst having been removed from the slurry 408 is then fed to a water-solubles separation unit 409 following the reaction wherein the water-soluble products 411 are removed from the slurry. The method used in the water-solubles separation step can be any of several methods known in the art, e.g. a water wash 410 or a counter current water wash wherein the reaction products are contacted with water to form a water phase comprising the water soluble components of the reaction slurry. Those skilled in the art would recognize that other separation methods would be suitable for this step. Water-soluble products can include, for example, glycerol and glycerol derivatives e.g. glycerol phosphatidyls.

In alternative embodiments, the reaction slurry (wherein the ammonia and, if present, the catalyst have been removed) is transferred to a static mixer wherein it is mixed with water. The water and reaction slurry mixture is then transferred to a decanter wherein an oil (lipid) stream comprising the fatty amides and any unreacted feedstock, and an aqueous stream are formed and are separated.

The aqueous stream (comprising glycerol and any derivatives thereof) is then transferred to a glycerol stripping column, e.g. a 4-stage stripping column, in which the aqueous stream is introduced to the top of the column and, upon contacting the bottom of the column is heated such that a vapor phase is generated and rises to the top of the column where it is removed. In this exemplary embodiment, the column “bottoms” are a primarily a glycerol product that can be, for example, in the range about 80 to 88 wt % glycerol, e.g. about 85% glycerol.

The remaining water-insoluble products 412 (e.g., a lipid stream resulting from the water-washing step) from the reaction slurry are then transferred to an amide separation unit 413 wherein the various components are separated into discreet fraction, e.g. amides 414 and “bottoms” (such as glycerides (acylglycerols) and/or unreacted feedstock) 415. The method for this step can be any of several known in the art, for example distillation, crystallization or the like. In certain embodiments, amides are separated from the reaction slurry by recrystallization using a solvent. Optionally, unreacted feedstock 416 can be recycled for subsequent reactions 403 following the separation step.

Ammonolysis of Phospholipids Using a Primary or Secondary Amine:

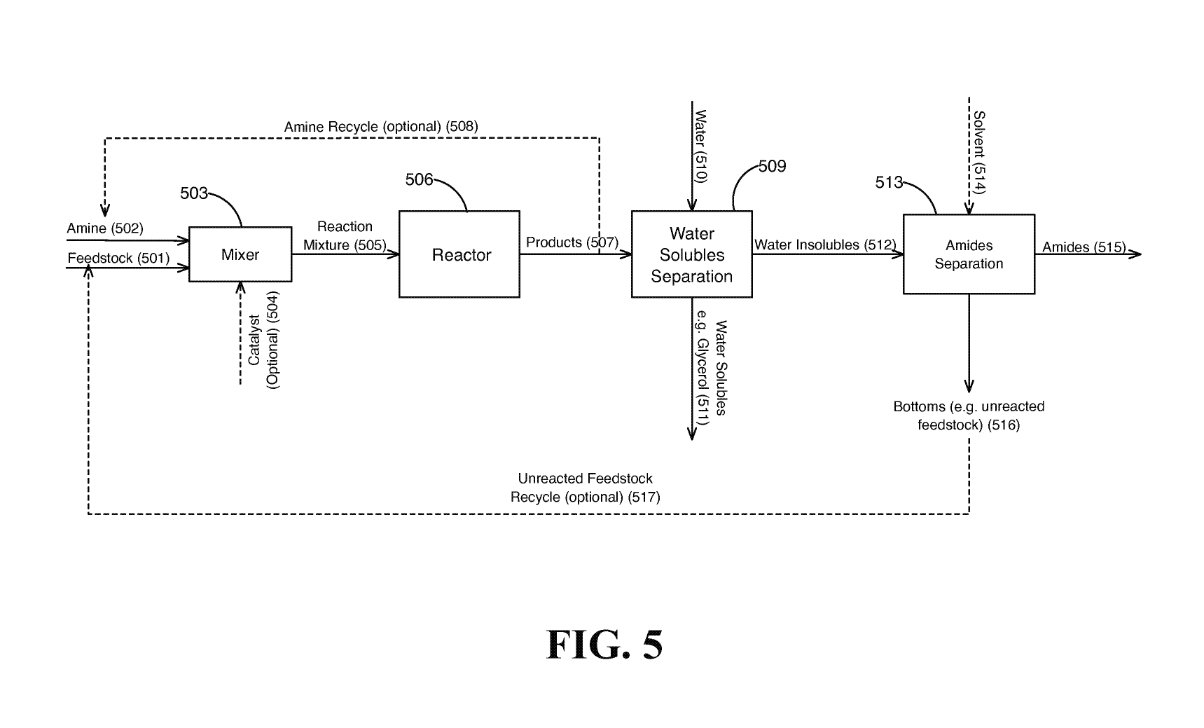

Referring to the exemplary method as set forth in FIG. 5, certain embodiments provide for the generation of fatty amides from a feedstock comprising phospholipids initiated by mixing the feedstock 501 with primary or secondary amine 502, e.g. mono-ethanolamine or diethanolamine, using any suitable mixer 503 and feeding the “reaction” mixture 505 to a reactor 506 and subjecting it to temperatures and pressures sufficient to allow for ammonolysis, thereby cleaving the fatty acids from the phospholipids and generating products 507, such as fatty amides and glycerol-phosphatidyls and generating products from the reaction 506. If present in the feedstock, all or a portion of the glycerides can be subjected to ammonolysis in the same reaction step, thereby cleaving the fatty acids from the glycerides and generating glycerol and fatty amides. Optionally, an ammonium salt catalyst 504 is added to the reaction mixture, e.g. ammonium chloride.

The primary amine can be, for example and without limitation, an aliphatic amine (e.g. methylamine, n-ethylamine, n-propylamine, n-butylamine, n-pentylamine, n-hexylamine, n-heptylamine, n-octylamine, n-nonylamine, n-decylamine, n-undecylamine, n-dodecylamine, n-tridecylamine, n-myristylamine, n-pentadecylamine, n-palmitylamine, n-heptadecylamine, n-stearamine, n-nonadecylamine, or n-eicosylamine) an alkanolamine, (e.g., alkanolamine: monoethanolamine, monopropanolamine, monobutanolamine, monopentanolamine, monohexanolamine, monoheptanolamine, monooctanolamine, monononanolamine, monodecanolamine, monoundecanolamine, monododecanolamine, monotridecanolamine, monobutadecanolamine, monopentadecanolamine, monohexadecanolamine, monoheptadecanolamine, monooctadecanolamine, monononadecanolamine, or monoeicosanolamine) a branched chain amine (e.g. isopropylamine, isobutylamine, isopentylamine, isohexylamine, isoheptylamine, isooctylamine, isononylamine, isodecylamine, sec-butylamine, sec-pentylamine, sec-hexylamine, sec-heptylamine, sec-octylamine, sec-nonylamine, sec-decylamine, tert-butylamine, tert-pentylamine, tert-hexylamine, tert-heptylamine, tert-octylamine, tert-nonylamine, or tert-decylamine), an ether amine (e.g. methoxyethylamine, ethoxyethylamine, methoxypropylamine, ethoxypropylamine, methoxybutylamine, ethoxybutylamine, methoxypentylamine, ethoxypentylamine, methoxyhexylamine, ethoxyhexylamine, methoxyheptylamine, ethoxyheptylamine, methoxyoctylamine, or ethoxyoctylamine), a cyclic amine (e.g. cyclohexylamine, cyclopentylamine, cyclobutylamine, cyclopropylamine, cycloheptylamine, cyclooctylamine, cyclononylamine, or cyclodecylamine), or an aryl amine (e.g. Aryl: benzylamine, phenylethylamine, aniline, toluidine, or anisidine).

The secondary amine can be, for example and without limitation, an aliphatic amine (e.g. dimethylamine, diethylamine, dipropylamine, dibutylamine, dipentylamine, dihexylamine, diheptylamine, dioctylamine, dinonylamine, didecylamine, diundecylamine, didodecylamine, ditridecylamine, dimyristylamine, dipentadecylamine, dipalmitylamine, diheptadecylamine, distearylamine, dinonadecylamine, dieicosylamine, ethylenediamine, propylenediamine, butylenediamine, pentylenediamine, hexylenediamine, heptylenediamine, octylenediamine, nonylenediamine, or decylenediamine) an alkanolamine, (e.g. Alkanolamine: diethanolamine, dipropanolamine, dibutanolamine, dipentanolamine, dihexanolamine, diheptanolamine, dioctanolamine, dinonanolamine, didecanolamine, diundecanolamine, didodecanolamine, ditridecanolamine, dibutadecanolamine, dipentadecanolamine, dihexadecanolamine, diheptadecanolamine, dioctadecanolamine, dinonadecanolamine, or dieicosanolamine) a branched chain amine (e.g. diisopropylamine, diisobutylamine, diisopentylamine, diisohexylamine, diisoheptylamine, diisooctylamine, diisononylamine, diisodecylamine, di-sec-butylamine, di-sec-pentylamine, di-sec-hexylamine, di-sec-heptylamine, di-sec-octylamine, di-sec-nonylamine, di-sec-decylamine, di-tert-butylamine, di-tert-pentylamine, di-tert-hexylamine, di-tert-heptylamine, di-tert-octylamine, di-tert-nonylamine, or di-tert-decylamine), an ether amine (e.g. dimethoxyethylamine, diethoxyethylamine, dimethoxypropylamine, diethoxypropylamine, dimethoxybutylamine, diethoxybutylamine, dimethoxypentylamine, diethoxypentylamine, dimethoxyhexylamine, diethoxyhexylamine, dimethoxyheptylamine, diethoxyheptylamine, dimethoxyoctylamine, or diethoxyoctylamine), a cyclic amine (e.g. dicyclohexylamine, dicyclopentylamine, dicyclobutylamine, dicyclopropylamine, dicycloheptylamine, dicyclooctylamine, dicyclononylamine, or dicyclodecylamine), or an aryl amine (e.g. Aryl: dibenzylamine, diphenylethylamine, dianiline, ditoluidine, or dianisidine).

The amount of primary or secondary amine in the reaction mixture can be in the range of between about 0 to about 100 mol per mol of feedstock, e.g. between about 1 mol to about 80 mol per mol of feedstock, about 2 mol to about 60 mol per mol of feedstock, about 3 mol to about 40 mol per mol of feedstock, about 4 mol to about 20 mol per mol of feedstock, or about 5 mol to about 10 mol per mol of feedstock. If present in the reaction mixture, the amount of catalyst can be in the range of between about 0 mol to about 10 mol per mol of feedstock, e.g. about 0 mol to about 8 mol per mol of feedstock, about 0 mol to about 6 mol per mol of feedstock, about 0 mol to about 4 mol per mol of feedstock, or about 0 mol to about 2 mol per mol of feedstock.

The temperature of the reactor can be in the range of between about 10° C. to about 175° C., e.g. between about 40° C. and about 120° C. or between about 75° C. and about 90° C. The pressure of the reactor can be in the range of between about 0 psi and about 2500 psi; or, between about 500 psi and about 2000 psi, between about 800 psi and about 1750 psi, or about 1000 psi and about 1500 psi; or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi. In certain embodiments, the reaction is carried out at ambient pressure. The reaction time can be in the range of between about 0 to about 12 hours, e.g. between about 1 hour and about 10 hours, between about 90 minutes and about 8 hours, between about 2 hours and 6 hours, or between about 3 hours and 5 hours.

For example, in an exemplary amidation reaction, where the solvent is an ammonia derivative, e.g. a primary amine or a secondary amine, the amount of feedstock used is between about 5-50% of the total weight of the reaction mixture, the amount of ammonia derivative used is between about 5-50% of the total weight of the total weight of the reaction mixture, the temperature is between about 80-140° C., the pressure is between about 0-100 psig, and the reaction time is between about 1-12 hr.

Following the reaction, in certain embodiments, un-reacted primary or secondary amine can be separated from the reaction slurry and recycled 508 for use in subsequent reactions 506. Several techniques are suitable for this step and is would be understood by those in the art that any of these techniques may be applied. In alternative embodiments, un-reacted primary or amine is separated from the reaction slurry using a vacuum evaporation method. In such embodiments, the reaction slurry is transferred from the reactor to a distillation column in which substantially all of the unreacted primary or secondary amine, e.g. 99% or more of the unreacted primary or secondary amine, is removed. The conditions in the distillation column will vary depending on the amine used for the reaction. In alternative embodiments in which it is an object of the process to separate any unreacted feedstock from the reaction slurry (which can then be recycled for subsequent reactions) the temperature in the distillation column should not exceed the temperature above which the gums in the unreacted feedstock will begin to degrade, e.g. approximately 145° C.

For example, the boiling point of ethylenediamine is 116° C. at atmospheric pressure, which is below the degradation temperature (145° C.) of the gums in the unreacted feedstock. Accordingly, the distillation process to remove unreacted amine from the reaction mixture can be conducted at atmospheric pressure. Alternatively, the boiling point of monoethanolamine is 178° C. at atmospheric pressure. Therefore, if monoethanolamine is the amine used in the reaction, and it is recovered in an amine recovery step, the pressure in the distillation column used to recover the monoethanolamine should be reduced to about less than 0.5 atm (14.7 psi) so that the monoethanolamine can be boiled off without degrading the gums in the unreacted feedstock.

In certain embodiments, the reaction slurry can be fed to a water-solubles separation unit 509 following the reaction wherein the water-soluble products (e.g., glycerol) 511 are removed from the slurry. The method used in the water-solubles separation step can be any of several methods known in the art, e.g. a water wash or a counter-current water wash wherein water the slurry is contacted with water 510 to generate a water phase comprising the water-soluble components of the reaction mixture. Those skilled in the art would recognize that other separation methods would be suitable for this step. Water-soluble products can include, for example, glycerol and glycerol derivatives e.g. glycerol phosphatidyls.

In alternative embodiments, the reaction slurry (wherein the unreacted primary or secondary amine have been removed) is transferred to a static mixer wherein it is mixed with water. The water and reaction slurry mixture is then transferred to a decanter wherein an oil (lipid) stream comprising the fatty amides and any unreacted feedstock, and an aqueous stream are formed and are separated.

The aqueous stream (comprising glycerol and any derivatives thereof) is then transferred to a glycerol stripping column, e.g. a 4-stage stripping column, in which the aqueous stream is introduced to the top of the column and, upon contacting the bottom of the column is heated such that a vapor phase is generated and rises to the top of the column where it is removed. In this exemplary embodiment, the column “bottoms” are a primarily a glycerol product that can be, for example, in the range about 80 to 88 wt % glycerol, e.g. about 85% glycerol.

The remaining water-insoluble products from the reaction slurry 512 are then transferred to an amides separation unit 513 wherein the various components are separated into discreet fraction, e.g. amides 515 and glycerides and/or any unreacted feedstock (so-called “bottoms”) 516. The method for this step can be any of several known in the art, for example distillation, crystallization or the like. In certain embodiments, amides are separated from the reaction slurry by recrystallization using a solvent 514. If separated, the unreacted feedstock can optionally be recycled 517 for use in subsequent reactions 506.

The amides produced in any embodiment of the present invention can then be used directly as end products, or as intermediates for subsequent upgrading steps. Fatty amides can be isolated (e.g. oleamide, stearamide, erucamide, behenamide, n-oleylpalmitamide, n-stearylerucamide, etc) and used directly as lubricants, as building blocks for the production of synthetic resins (e.g. polyethylene, polypropylene, etc.), anti-blocking agents, printing ink additives, and pigments and dye dispersants. Potential upgrading steps include, without limitation, dehydration to generate nitriles, direct hydrogenation to generate amines, or as an intermediate in the production of isocyanates.

The fatty acids produce in any embodiment of the present invention can be used and/or processed for use in a variety of industrial applications. For example, the fatty acids can be purified and/or separated into individual fatty acids using any suitable technique or method known in the art, e.g. pressing, solvent crystallization, fractional distillation, or the like. The fatty acids can also be converted to various products through additional reaction steps. These include, without limitation, hydrogenation, oxidative cleavage, polymerization, and the like. Uses of fatty acids and their derivatives include:
Cosmetics and toiletries: can be used directly or, for example, saponified for use in a range of cosmetic and toiletry ingredients such as shampoos, shaving creams etc.
Foods: fatty acids produced in alternative embodiments of the present invention can be used directly or upgraded and/or converted for use as emulsifiers, stabilizers, surface active agents, lubricants, and plasticizers in food products.
Diacids (Dicarboxylic acids): The fatty acids produced in any of the embodiments of the present invention can be converted to diacids (dicarboxylic acids) using any suitable method known in the art. The diacids produced can be used in a range of industrial applications, e.g. as adhesives, plasticizers, gelatinizing agents, hydraulic fluids, lubricants, emollients, and for the production of polymers e.g. nylon and polyurethane foam.
Azelaic acid: A specific diacid, azelaic acid, can be produced, for example, by reacting an oleic acid produced in any embodiment of the present invention with chromic acid, resulting in the oxidative cleavage of the oleic acid to produce azelaic acid, or by the ozonolysis of oleic acid, or any other suitable method known in the art
Other industrial applications including ingredients in the production of soaps and synthetic detergents, wetting agents in textile manufacturing, industrial lubricating greases and oils, paints and protective coatings, and as vulcanizing and compounding agents in the production of natural rubber.

The invention will be further described with reference to the following examples; however, it is to be understood that the invention is not limited to such examples.

### EXAMPLES

### Example 1

### Production of Primary Fatty Amides from a Soy Lecithin Feedstock

This example describes an exemplary process of the invention for the production of primary fatty amides from a soy lecithin feedstock.

First, 31.7 g of ammonium acetate and 320 g of YELKIN-T® (Yelkin-T®) lecithin were placed in a 1900 mL Parr autoclave, sealed, and brought to 110° C. while under nitrogen flush to provide a substantially anhydrous reaction system. Once all visible moisture was removed, the autoclave was sealed off and transferred to a dry ice bath, wherein the lower half of the autoclave was submerged in the ice bath. The autoclave was cooled in dry ice bath for 1 hr. Next, 300 mL of commercial anhydrous ammonia were condensed into a graduated cold trap that had been flushed with nitrogen for 1 hr prior to the ammonia condensation procedure. A vacuum was pulled on the autoclave via ⅛″ metering valve, then closed to contain the negative pressure. Liquid ammonia was transferred into the autoclave via a negative pressure cannula attached to a separate ⅛″ metering valve on the autoclave. The autoclave was then sealed and heated to 125° C. and reacted for 4 hrs. Post reaction, ammonia gas is carefully purged into a neutralizing solution. The autoclave was kept at 125° C. to ensure that contents remained molten for easy handling.

The reaction contents were then poured into an equal volume of mixing water in order to remove glycerol and its derivatives, phosphoric acid and its derivatives, and the catalyst. Solid particulates from the reaction were gravity filtered through a 20 micron bag filter, and freeze dried. The forgoing reaction resulted in a 50 wt % yield of fatty amides, or 160 grams of fatty amides.

The primary amides from the foregoing reaction were then analyzed using gas chromatography (GC) to determine the ratio of individual amides produced, based on the chemical structure as determined by their fatty acid group. The results of the GC analysis are shown in Table 1.

TABLE 1

| | | Relative % of primary amide |
|---|---|---|
| Primary amide | Molecular | (wt % amide/wt % total |
| (fatty acid structure) | Weight | amides in product mixture) |
| 18:3 | 277.5 | 6 |
| 18:2 | 279.5 | 56 |
| 18:1 | 281.5 | 23 |
| 18:0 | 283.5 | 3 |
| 16:0 | 255.4 | 11 |

### Example 2

### Production of Secondary Fatty Amides from a Soy Lecithin Feedstock

This example describes an exemplary process of the invention for the production of secondary fatty amides from a soy lecithin feedstock.

First, 19.7 g of monoethanolamine (MEA) and 25 g of YELKIN-T® (Yelkin-T®) lecithin were placed in a 250 mL single-neck round bottom with a condenser under a nitrogen flush to provide a substantially anhydrous reaction system. Once all visible moisture was removed, the round bottom was heated, using an oil bath, to 90° C. and reacted for 3 hrs. The conversion of the lecithin and production of ethanolamide was monitored using thin-layer chromatography (TLC). While molten, the reaction contents were poured into boiling water to separate the ethanolamides from excess MEA and water-soluble byproducts. Washed ethanolamides were filtered with a Buchner funnel and coarse filter paper. The filtered contents were then freeze dried to gather a dry mass.

The forgoing reaction resulted in a (freeze dried) yield of 19.7 g ethanolamide.

The ethanolamides from the foregoing reaction were then analyzed using gas chromatography (GC) to determine the ratio of individual amides produced, based on the chemical structure as determined by their fatty acid group. The results of the GC analysis are shown in Table 1.

TABLE 2

| | | Relative % of Amide |
|---|---|---|
| Amide | Molecular | (wt % amide/wt % total |
| (fatty acid structure) | Weight | amides in product mixture) |
| 18:3 | 321.6 | 6 |
| 18:2 | 323.6 | 56 |
| 18:1 | 325.6 | 23 |
| 18:0 | 327.6 | 3 |
| 16:0 | 299.5 | 11 |

While the forgoing written description of the invention enables one of ordinary skill to make and use what is considered presently to be the best mode thereof, those of ordinary skill will understand and appreciate the existence of variations, combinations, and equivalents of the specific embodiments, methods, and examples herein. The invention should therefore not be limited by the above described embodiments, methods and examples, but by all embodiments and methods within the scope and spirit of the invention.

1. A process for the production of free fatty acids and/or free fatty acid derivatives, from a lipid-containing feedstock comprising phospholipids comprising the steps of:
a. providing a feedstock comprising phospholipids,
wherein optionally the feedstock further comprises non-phospholipid lipids or, lipids other than phospholipids, and optionally the non-phospholipid lipids comprise glycerides, and optionally the glycerides are triglycerides;
b. providing a reaction mixture comprising the feedstock and a solvent and, optionally, CO₂; and
c. reacting the reaction mixture at a temperature, pressure and for a time that is sufficient to provide for the solvolysis, esterification, transesterification, acidification, or any combination thereof, of substantially all of the phospholipids and, if present, the lipids in the feedstock,
wherein optionally the time is between about 0 to about 24 hours, between about 0 to about 12 hours, or between about 1 hour and about 8 hours, between about 3 hours and 5 hours, between about 5 minutes and one hour, between about 2 minutes to about 20 minutes, or between about 0 minutes to about 1 minute,
wherein optionally the solvolysis is an alcoholysis, an ammonolysis, a hydrolysis, or a combination thereof.

2. The process of claim 1, wherein:
(a) the feedstock comprises from between about 1 to 10 wt % to about 90 to 100 wt % phospholipids, or from between about 10 to 20 wt % to about 80 to 100 wt % phospholipids, about 30 wt % to about 90 wt %, about 40 wt % to about 80 wt %, or about 50 wt % to about 75 wt % phospholipids;
(b) the feedstock further comprises between about 5 wt % to about 40 wt % free fatty acids, and between about 5 wt % to about 50 wt % glycerides (acylglycerols);
(c) the feedstock is an oil gum derived from a crude vegetable oil, or a mixture of oil gums derived from multiple vegetable oils, and optionally the oil gum is derived from a crude vegetable oil selected from the group consisting of soybean oil, palm oil, canola oil, rapeseed oil, wheat germ oil, safflower oil, peanut oil, cottonseed oil, sunflower oil, grape seed oil, jatropha oil, palm kernel oil, coconut oil, olive oil, corn oil, hazelnut oil, linseed oil, rice bran oil, safflower oil, sesame oil, algae oil and a combination thereof;
(d) the reaction temperature is between about 10° C. to about 175° C., the pressure of the reaction is between about 0 psi and about 2500 psi, or between about 500 psi and about 2000 psi, between about 800 psi and about 1750 psi, or about 1000 psi and about 1500 psi, or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi, and the time of the reaction is between about 0 to about 12 hours;
(e) the solvent is water, and optionally the reaction mixture comprises a CO₂ that reacts with the water to form carbonic acid in the reaction mixture, and optionally the amount of water in the reaction mixture is between about 1 mol to about 100 mol per mol of feedstock, or between about 10 mol to about 70 mol per mol of feedstock, or about 30 mol to about 50 mol per mol of feedstock, and optionally the amount of CO₂ in the reaction mixture is between about 1 mol to about 10 mol per mol of feedstock;
(f) the reaction mixture further comprises an alcohol, and wherein the alcohol esterifies at least a portion of the lipids in the feedstock to generate fatty alkyl esters, and optionally the amount alcohol in the reaction mixture is between about 1 mol to about 100 mol per mol of feedstock, or between about 10 mol to about 70 mol per mol of feedstock, or about 30 mol to about 50 mol per mol of feedstock, and optionally the alcohol contains between 1 and 9 carbon atoms, or the alcohol is a methanol, an ethanol, a butanol, an isopropyl alcohol, a sec-butanol, a t-butanol, a benzyl alcohol, or a combination thereof;
(g) the solvent is ammonia, and optionally the amount of ammonia in the reaction mixture is between about 1 mol to about 100 mol per mol of feedstock, or between about 10 mol to about 70 mol per mol of feedstock, or about 30 mol to about 50 mol per mol of feedstock, and optionally the reaction mixture further comprises a catalyst, and optionally the catalyst is an ammonium salt, and optionally the catalyst is selected from the group consisting of ammonium nitrate, ammonium acetate, acetic acid, sodium acetate, sodium methoxide, sodium ethoxide and a combination thereof;
(h) the catalyst is glycerol; or
(i) the solvent is a primary or a secondary amine, and optionally the primary amine is an aliphatic amine, an alkanolamine, a branched chain amine, an ether amine, a cyclic amine, an aryl amine, or any combination thereof, and optionally the amount of primary amine in the reaction mixture is between about 1 mol to about 100 mol per mol of feedstock, or between about 10 mol to about 70 mol per mol of feedstock, or about 30 mol to about 50 mol per mol of feedstock.

3-22. (canceled)

23. A process for the production of free fatty acids from a lipid-containing feedstock comprising phospholipids and, optionally, other lipids or glycerides, the process comprising:
a. providing a reaction mixture that comprises the feedstock, a solvent comprising water, and CO₂, wherein the water and CO₂ react to form carbonic acid; and
b. reacting the reaction mixture at a temperature and pressure and for a time that is sufficient to provide for the alcoholysis or esterification and/or acidification of substantially all of the phospholipids and, if present, the other lipids in the feedstock,
wherein optionally the time is between about 0 to about 24 hours, between about 0 to about 12 hours, or between about 1 hour and about 8 hours, between about 3 hours and 5 hours, between about 5 minutes and one hour, between about 2 minutes to about 20 minutes, or between about 0 minutes to about 1 minute.

24. The process of 23, wherein:
(a) the feedstock comprises from between about 10 wt % to about 100 wt % phospholipids, or from between about 20 wt % to about 100 wt % phospholipids, about 30 wt % to about 90 wt %, about 40 wt % to about 80 wt %, or about 50 wt % to about 75 wt % phospholipids;
(b) the feedstock further comprises between about 5 wt % to about 40 wt % free fatty acids, and between about 5 wt % to about 50 wt % glycerides.
(c) the feedstock is an oil gum derived from a crude vegetable oil or a mixture of oil gums derived from multiple vegetable oils, and optionally the crude vegetable oil is selected from the group consisting of soybean oil, palm oil, canola oil, rapeseed oil, wheat germ oil, safflower oil, peanut oil, cottonseed oil, sunflower oil, grape seed oil, jatropha oil, palm kernel oil, coconut oil, olive oil, corn oil, hazelnut oil, linseed oil, rice bran oil, safflower oil, sesame oil, algae oil, and a combination thereof;
(d) the reaction temperature is between about 10° C. to about 175° C., the pressure of the reaction is between about between about 0 psi and about 2500 psi, or between about 500 psi and about 2000 psi, between about 800 psi and about 1750 psi, or about 1000 psi and about 1500 psi, or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi, and the time of the reaction is between about 0 to about 12 hours;
(e) the amount of water in the reaction mixture is between about 1 mol to about 100 mol per mol of feedstock, or between about 10 mol to about 70 mol per mol of feedstock, or about 30 mol to about 50 mol per mol of feedstock;
(f) the amount of CO₂ in the reaction mixture is between about 1 mol to about 10 mol per mol of feedstock;
(g) the reaction mixture further comprises an alcohol, and wherein in the reaction the alcohol esterifies at least a portion of the lipids in the feedstock to generate fatty alkyl esters; or
(h) the amount of alcohol in the reaction mixture is between about 1 mol to about 100 mol per mol of feedstock, or between about 10 mol to about 70 mol per mol of feedstock, or about 30 mol to about 50 mol per mol of feedstock, and optionally the alcohol contains between 1 and 9 carbon atoms, or the alcohol is a methanol, an ethanol, a butanol, an isopropyl alcohol, a sec-butanol, a t-butanol, a benzyl alcohol, or a combination thereof.

25-33. (canceled)

34. A process for the production of fatty amides from lipid feedstocks comprising phospholipids and, optionally, other lipids, or glycerides, the process comprising:
a. providing a reaction mixture that comprises the feedstock, a solvent comprising an ammonia, and a catalyst; and
b. reacting the reaction mixture at a temperature and pressure and for a time that is sufficient to provide for the solvolysis of substantially all of the phospholipids and, if present, the other lipids in the feedstock,
wherein optionally the time is between about 0 to about 24 hours, between about 0 to about 12 hours, or between about 1 hour and about 8 hours, between about 3 hours and 5 hours, between about 5 minutes and one hour, between about 2 minutes to about 20 minutes, or between about 0 minutes to about 1 minute.

35. The process of 34, wherein:
(a) the feedstock comprises from between about 10 wt % to about 100 wt % phospholipids, or from between about 20 wt % to about 100 wt % phospholipids, about 30 wt % to about 90 wt %, about 40 wt % to about 80 wt %, or about 50 wt % to about 75 wt % phospholipids;
(b) the feedstock further comprises between about 5 wt % to about 40 wt % free fatty acids, and between about 5 wt % to about 50 wt % glycerides;
(c) the feedstock is an oil gum derived from a crude vegetable oil, and optionally the oil gum is derived from a crude vegetable oil selected from the group consisting of a soybean oil, palm oil, canola oil, rapeseed oil, wheat germ oil, safflower oil, peanut oil, cottonseed oil, sunflower oil, algae oil, or a combination thereof;
(d) the reaction temperature is between about 10° C. to about 175° C., the pressure of the reaction is between about 0 psi and about 2500 psi, or between about 500 psi and about 2000 psi, between about 800 psi and about 1750 psi, or about 1000 psi and about 1500 psi, or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi, and the time of the reaction is between about 0 to about 12 hours;
(e) the amount of ammonia in the reaction mixture is between about 1 mol to about 100 mol per mol of feedstock, or between about 10 mol to about 70 mol per mol of feedstock, or about 30 mol to about 50 mol per mol of feedstock;
(f) catalyst is an ammonium salt, and optionally the catalyst is selected from the group consisting of ammonium nitrate, ammonium acetate, acetic acid, sodium acetate, sodium methoxide, sodium ethoxide and a combination thereof, or
(g) the catalyst is glycerol.

36.-43. (canceled)

44. A process for the production of fatty amides from lipid feedstocks comprising phospholipids and, optionally, other lipids, or glycerides, the process comprising:
a. providing a reaction mixture that comprises the feedstock and a solvent comprising a primary amine or a secondary amine, and optionally, a catalyst; and
b. reacting the reaction mixture at a temperature and pressure and for a time that is sufficient to provide for the solvolysis of substantially all of the phospholipids and, if present, the other lipids in the feedstock.
wherein optionally the time is between about 0 to about 24 hours, between about 0 to about 12 hours, or between about 1 hour and about 8 hours, between about 3 hours and 5 hours, between about 5 minutes and one hour, between about 2 minutes to about 20 minutes, or between about 0 minutes to about 1 minute.

45. The process of 44, wherein:
(a) the feedstock comprises between about 10 wt % to about 100 wt % phospholipids;
(b) the feedstock further comprises between about 5 wt % to about 40 wt % free fatty acids, and between about 5 wt % to about 50 wt % glycerides;
(c) the feedstock is an oil gum derived from a crude vegetable oil, and optionally the oil gum is derived from a crude vegetable oil selected from the group consisting of soybean oil, palm oil, canola oil, rapeseed oil, wheat germ oil, safflower oil, peanut oil, cottonseed oil, sunflower oil and algae oil;
(d) the reaction temperature is between about 10° C. to about 175° C., the pressure of the reaction is between about 0 psi and about 2500 psi, or between about 500 psi and about 2000 psi, between about 800 psi and about 1750 psi, or about 1000 psi and about 1500 psi, or between about 0 psi and about 2000 psi, between about 0 psi and about 1500 psi, between about 0 psi and about 1000 psi, between about 0 psi and about 500 psi, between about 0 psi and about 100 psi, between about 0 psi and 50 psi, between about 0 psi and about 30 psi, or about 0 psi and about 10 psi, and the time of the reaction is between about 0 to about 12 hours;
(e) the amount of primary amine in the reaction mixture is between about 1 mol to about 100 mol per mol of feedstock, or between about 10 mol to about 70 mol per mol of feedstock, or about 30 mol to about 50 mol per mol of feedstock;
(f) the amount of catalyst in the reaction mixture is between about 1 mol to about 10 mol per mol of feedstock;
(g) the catalyst is an ammonium salt, or the catalyst is selected from the group consisting of ammonium nitrate, ammonium acetate, acetic acid, sodium acetate, sodium methoxide, sodium ethoxide and a combination thereof, or the catalyst comprises a glycerol; or
(h) the primary amine is an aliphatic amine, an alkanolamine, a branched chain amine, an ether amine, a cyclic amine, an aryl amine, or any combination thereof.

46.-55. (canceled)
